(19) United States
(12) Patent Application Publication
(10) Pub. No.: US 2023/0123464 A1
(43) Pub. Date: Apr. 20, 2023

(54) METHODS FOR DIAGNOSING EXPOSURE TO COLORECTAL CANCER CARCINOGENS USING BIOLOGICAL SAMPLES

(71) Applicant: GOODBEING CENTER CO., LTD., Seoul (KR)

(72) Inventor: Mihi YANG, Seoul (KR)

(73) Assignee: GOODBEING CENTER CO., LTD., Seoul (KR)

(21) Appl. No.: 17/790,951

(22) PCT Filed: Jan. 4, 2021
(86) PCT No.: PCT/KR2021/000016

§ 371 Date: Oct. 26, 2022

(30) Foreign Application Priority Data
Jan. 8, 2020 (KR) 10-2020-0002492

(51) Int. Cl. G01N 33/574; G01N 1/40; G01N 30/72

(52) U.S. Cl. CPC G01N 33/57419; G01N 1/4055; G01N 30/7233; G01N 2001/4061; G01N 2030/062

(57) ABSTRACT

The present invention relates to a method for providing information for the preemptive diagnosis of colorectal cancer with non-invasive biospecimens, wherein the excellent quantification of carcinogenic PhIP and MeIQx is achieved in terms of yields, when a biological sample, such as urine, is hydrolyzed with strong alkali reagents and then prepared through a specific liquid-liquid extraction, as compared to when the sample is prepared with other hydrolysis methods or extraction.

### TECHNICAL FIELD

The present disclosure is regard to the analysis methods to provide exposure levels of carcinogens of colorectal cancer with non-invasive biospecimens to prevent from colorectal cancer

### BACKGROUND ART

Colorectal cancer is a malignant tumor that occurs in the appendix, colon, and rectum, and is developed in the mucous membrane which is the innermost surface of the large intestine.

According to the Korean cancer statistics in 2006, it is the second most common cancer in Korea after gastric cancer, and its incidence has been steeply grown in recent years with the westernized diet habits. In the last 10 years, the mortality rate of colorectal cancer has been increased by about 80%, and the rate is continuously going up. Sixties are the most common age of the colorectal cancer incidence and more than 90% of colorectal cancer patients are over 40 s and the incidence rate doubles every 10 years. Most colorectal cancer cases start from benign polyps, which protrude after undergoing abnormal growth of the epithelium from the inner wall of the colon and are a very common disease that occurs in 15 to 20% of adults. A family history of colorectal cancer or chronic ulcerative colitis accelerate the risk of colorectal cancer. Moreover, colorectal cancer is one of major cancers that are related to diet, including low fiber intake, heavy intake of animal fat, or excessive intake of refined sugar due to westernized diet.

Most colorectal cancer is diagnosed by the detection of cancer cells with colonoscopy and has no symptoms at an early stage, thereby the early diagnoses is quite difficult. In addition, there are very few non-invasive methods for predicting colorectal cancer. In addition, most of diagnostic methods for cancers are to detect solid cancers, such as colorectal cancer that have already progressed. Therefore, the prevention of colorectal cancer in the high susceptible people is efficient to save medical costs and death of colon cancer.

WHO/IARC recently stipulated processed meat and red meat as carcinogens (category 1, 2A, respectively). In addition, carcinogenic heterocyclic amines (HCAs), such as 2-amino-3,8-dimethylimidazo[4,5-f]quinoxaline (MeIQx) and 2-amino-1-methyl-6-phenylimidazo[4,5-b]pyridine (PhIP), are produced by cooking of meat (category 2B) and adduct to DNA to induce mutations. Therefore, it is necessary for cancer prevention to quantify carcinogenic HCAs accumulated in the body with non-invasive analyses using biospecimens.

### DISCLOSURE

### Technical Problem

There are very few non-invasive methods for predicting colorectal cancer. Therefore, the object of the present disclosure is to develop a specific and non-invasive method for analyses of carcinogenic HCAs in urine to provide information for cancer prevention.

### Technical Solutions

Example embodiments of the present disclosure includes collection of biospecimens from potential colorectal cancer patients, undergoing sample preparation with strong alkali-hydrolysis, liquid-liquid extraction, and analyses of the levels of carcinogenic HCAs, i.e., MeIQx and PhIP, with LC/MS/MS.

For the quantification step on LC/MS/MS, they reduce analytic errors, e.g., carry over in auto samplers, and improve accuracy and reproducibility.

### Advantageous Effects

The analytic yield of HCAs of the present disclosure, i.e., the alkali hydrolysis of urine and liquid-liquid extraction, is superior to those of other hydrolysis methods with enzymes or strong acids, therefore, the present disclosure provides preemptive, effective and non-invasive diagnosis for colorectal cancer.

### BRIEF DESCRIPTIONS OF THE DRAWINGS

FIG. 1 shows the results of colon cancer case-control studies for the levels of urinary MeIQx and PhIP in 218 subjects, i.e., 109 persons for each group, case and control group, respectively. There were positive relations between the levels of urinary MeIQx or PhIP and those of 1-hydroxypyrene (1-OHP), a biomarker for polycyclic aromatic hydrocarbons (PAHs), or malondialdehyde (MDA), a biomarker of oxidative stress, which were analyzed by Lee et αll’s method (J Ginseng Res. 2011 Sep; 35(3): 339-343, FIG. 1A, p= 0.001; B, p=0.093; C, p=0.009; D, p=0.0052). These relationships provide evidences that MeIQx and PhIP are diet-derived carcinogens by cooking or food processing. As MeIQx and PhIP are amines with nitrogen and different from 1-OHP, the compound of carbon and hydrogen, they are specific and meaningful biomarkers for the intake of nitrogen compound, i.e., proteins of red meat and processed meat.

FIG. 2 shows a scheme of preparation for different hydrolysis methods.

FIG. 3 shows the LC/MS/MS profiles of the two HCAs (MeIQx and PhIP) and internal standards (MeIQx-d₃ and PhIP-d₃). FIG. 3A presents MeIQx ; FIG. 3B, MeIQx-d₃ ; FIG. 3C, PhIP; FIG. 3D, PhIP-d₃. It shows the proper resolution of the present LC/MS/MS method.

FIG. 4 shows the calibration curves for MeIQx (8-2400 ng/L) in a solvent (blue, solid, ▲) and urine (orange, dotted, ● ), which were analyzed the above LC/MS/MS method (FIG. 3). Every analysis was evaluated with the linear calibration curves (R² >0.99).

FIG. 5 shows the calibration curves of PhIP (12.8-1584.5 ng/L) as like as MeIQx (FIG. 4.)

### BEST MODE

Hereinafter, the present disclosure will be described in detail.

Colorectal cancer is one of the five most common cancers in Korea. Recently, WHO/IARC (World Health Organization/International Agency for Research on Cancer) defines processed meat and red meat as carcinogens, as like as PhIP and MeIQx, which are heterocyclic amines (HCAs) and bioproduced carcinogens from cooking the meat. MeIQx and

PhIP have been confirmed as carcinogens in real experiments. When the levels of MeIQx and PhIP are steadily high in urine, it becomes cancer after latent period. Therefore, the internal dose of the two HCAs can be a biomarker for the prevention and diagnoses of colorectal cancer, however, there are difficulties in the analyses of the HCAs in biospecimens due to lack of convenience. To solve the difficulties, the inventors of the present disclosure developed the non-invasive tests for MeIQx and PhIP with small volume of urine, including sample preparation and LC/MS/MS analyses. The inventors confirmed higher reproducibility, accuracy, convenience and versatility in the present disclosure than other previous analysis methods.

An example embodiment of the present disclosure can provide diagnostic information for exposure levels of colorectal cancer-carcinogens and includes collecting a non-invasive biological sample from a subject; treating the collected sample with strong alkali and performing liquid-liquid extraction; and quantifying a level of the two HCAs in the liquid liquid-liquid extracted sample.

To provide the above information, the method can further include specific washing procedure for the sample injector with a special washing solution in order to improve accuracy and reproducibility and to avoid analysis failure due to the residues of the HCAs (carry over phenomenon)

The biological sample may be selected from urine, blood, saliva, or tissues. More preferably, it may be urine, but is not limited thereto, if the sample is non-invasive to human subjects.

The collected sample may be treated with the strong alkali in an amount of 5 to 15 parts by weight based on 100 parts by weight of the sample, and optimally, in an amount of 10 parts by weight based on 100 parts by weight of the sample, but the present disclosure is not limited thereto.

In an example embodiment of the present disclosure, when the strong alkali is added to the sample beyond the optimal content range, the yield may decrease due to decomposition of the target materials, thereby causing an analytic error. The treatment below the content range may also result in inaccurate quantification due to insufficient hydrolyses.

Optimally, the test material may be added with 100 µl of 10N NaOH as well as 126.75 µl/L of PhIP-d₃ and 125 µl/L of MeIQx-d₃ as internal standards (ISs) to 1 ml of urine sample of a subject, who may have colorectal cancer or be exposed to colon cancer-carcinogen, and then mixed, but is not limited thereto.

The strong alkali may be selected from the group consisting of sodium hydroxide (NaOH) or potassium hydroxide (KOH).

For the liquid-liquid extraction, dichloromethane (CH2Cl₂) can be used as an extraction solvent rather than other solvents due to high performance.

The term “liquid-liquid extraction” as used herein refers to the method, which is a method of separating a specific substance in a mixture from other substances by applying a solvent to a stock solution of a liquid mixture during the extraction operation. The characteristic of liquid-liquid extraction is that the stock solution and the solvent form two phases to separate the two phases by difference in specific polarity and thereby selectively transferring a desired substance, that is, a solute to a solvent phase for separation.

The HCAs may be at least one or two selected from the group consisting of 2-amino-1-methyl-6-phenylimidazo(4-5-b)pyridine (PhIP) and 2-amino-3,8-dimethylimidazo(4,5-f)quinoxaline (MeIQx).

Since a trace amount of the HCAs exists in biospecimens, the liquid-liquid extracted sample may be analyzed by advanced instruments, such as liquid chromatography-mass spectrometry (LC/MS/MS), but is not limited thereto.

To provide information for early diagnosis of colorectal cancer, the method may be predict that the risk of colorectal cancer is higher in the people with high levels of the HCAs than in the others.

The terms “PhIP” and “MeIQx” as used herein refer to the HCA-structured carcinogens bioproduced from cooking amino acids in red meat and musculoskeletal components, such as creatine/creatinine, at a high temperature of 120° C. or higher.

### MODES FOR CARRYING OUT INVENTION

Hereinafter, the following examples will be described in detail for better understanding of the present disclosure. As the following merely exemplifies the contents of the present disclosure, the scope of the disclosure is not limited to the following examples. The examples provide more complete explanation of the present disclosure to those with average knowledge in the industry.

### <Example 1> Strong Relationship Between HCAs and Colorectal Cancer

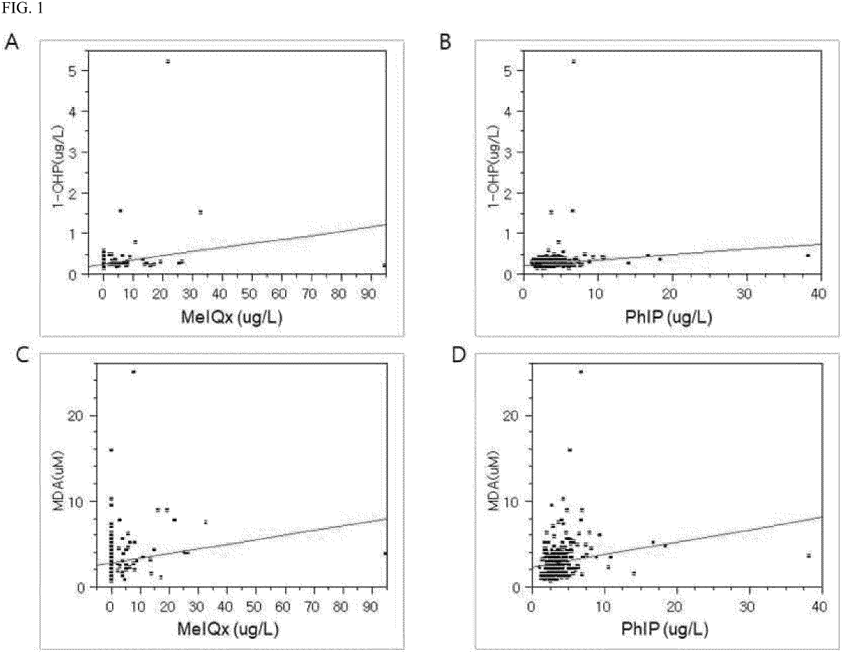

There were positive relations between the levels of urinary MeIQx or PhIP and those of 1-hydroxypyrene (1-OHP), a biomarker for polycyclic aromatic hydrocarbons (PAHs), or malondialdehyde (MDA), a biomarker of oxidative stress, which were analyzed by Lee et all’s method (J Ginseng Res. 2011 Sep; 35(3): 339-343 ; Cancer Epidemiol Biomarkers Prev. 2010 Mar; 19(3): 877-883.), among the urine samples gathered from colorectal cancer cases and controls (FIG. 1).

The urinary levels of MeIQx or PhIP in the cancer patients were higher than those in the normal controls.

The above results provide that MeIQx and PhIP are diet-derived carcinogens for colorectal cancer.

### <Example 2> Non-invasive Analyses With Urine

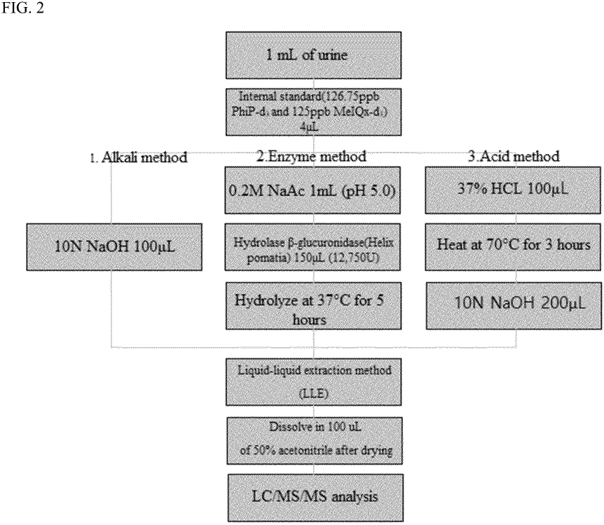

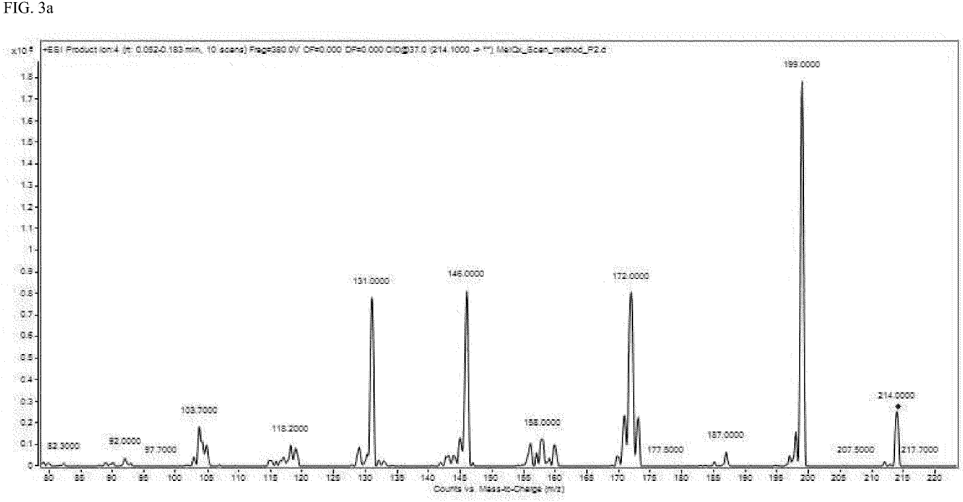

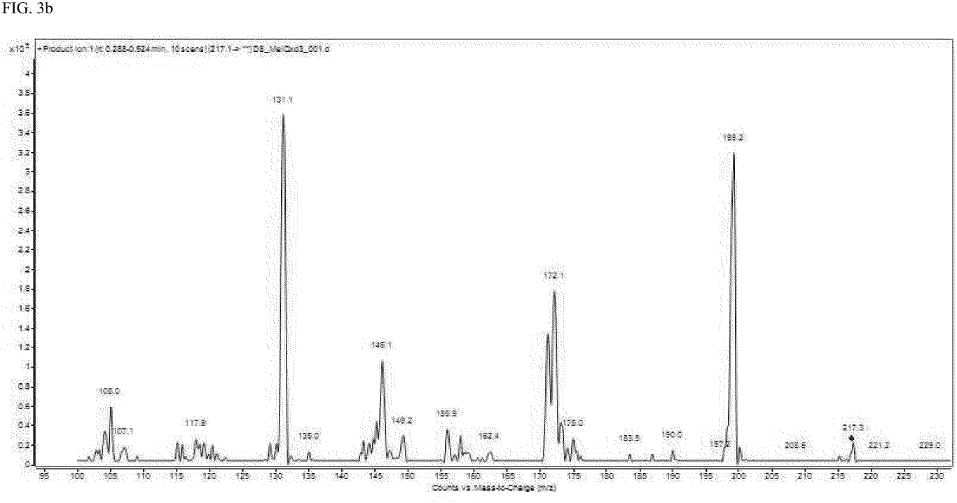

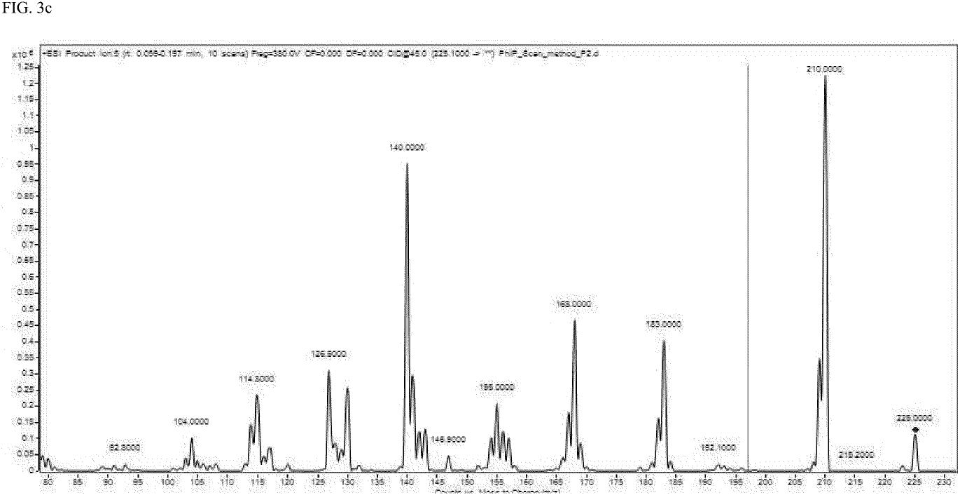

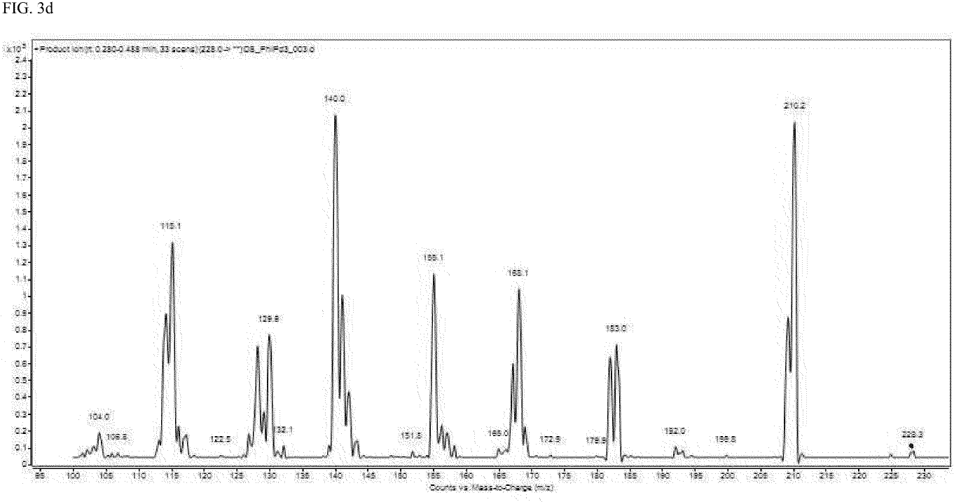

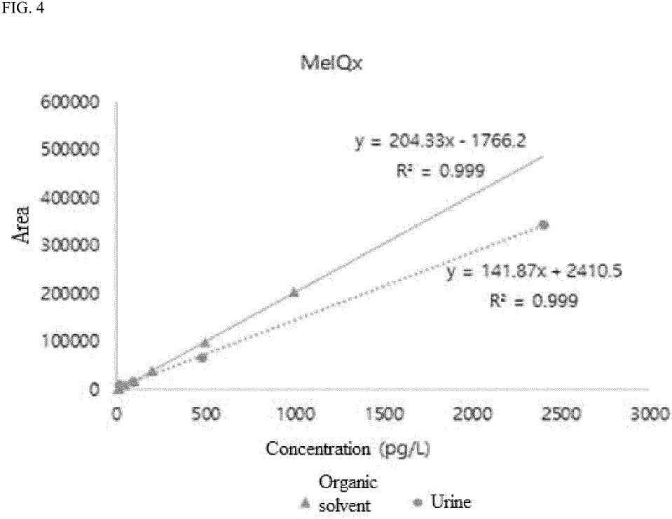

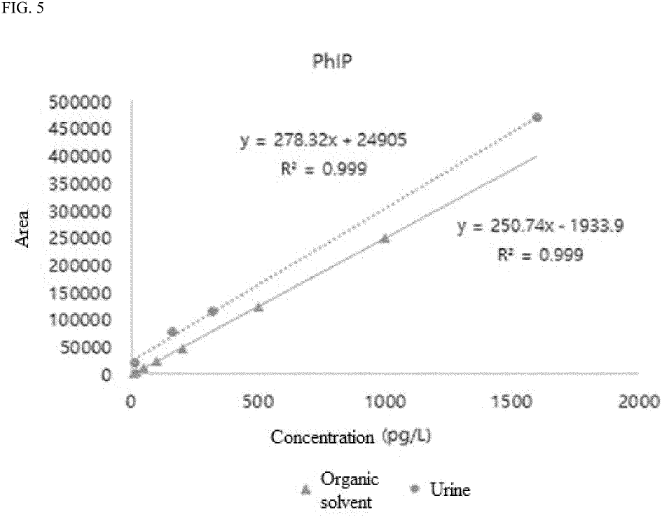

To estimate occurrence of colorectal cancer and to develop preemptive diagnoses for colorectal cancer, PhIP and MeIQx, biomarkers that reflect the intake of red or processed meat, carcinogens of colorectal cancer were effectively prepared in urine, a non-invasive biospecimen (FIG. 2) and tested with LC/MS/MS analytic systems for proper recovery rates.

### 1. Strong Alkali Method

One hundred µl of 10N NaOH and each 4 µl of internal standards, i.e., PhIP-d₃ (126.75 µg/L) and MeIQx-d₃(125 µg/L), were added to 1 mL of urine and mixed to be prepared as a test sample.

Thereafter, liquid-liquid extraction (LLE) was used to separate analytes from interfering substances in urine. For the extraction, 6 mL of dichloromethane (CH₂Cl₂) as an organic solvent and 10 mg of NaCl for salting out were added to the sample, followed by extraction with an extractor (EYELA CUTE MIXER CM-1000) for 10 minutes (speed 18) so as to facilitate extraction of the analyte in the urine to the solvent. After a pause, 5 mL of the CH₂Cl₂ layer in the lower layer was taken to a new test tube.

The above process was repeated twice. The total 10 mL of the CH₂Cl₂ layer was completely dried in a speed vac (Savant Automatic Environmental SpeedVac system AES1010). The dried samples were dissolved in 50% acetonitrile, filtered with a 0.2 µm filter, and analyzed with LC/MS/MS.

LC/MS/MS analyses were performed under the following conditions.
HPLC conditions
   a. HPLC system: Agilent 1290 Infinity Binary Pump, Column oven, Hip sampler
   b. Column: YMC Meteroic Core C18 (5.0×3.0 mm, 2.7 µm)
   c. Injection volume: 5 µl
   d. Column temperature: 40° C.
   e. Flow rate: 0.5 ml/min
   f. Mobile phase: A - 0.01% Formic acid, 20 mM Ammonium formate in distilled deionized water; B - Acetonitrile
   B-Acetonitrile
   g. Solvent gradient: 5% B (0 min) - 95% (7 min) → 95% (9.5 min) → 5% (10 min) → 5% (17 min)
   h. Injection needle wash: 5% NH₄OH + 47.5% methanol + 47.5% acetonitrile
2) MS conditions
   a. Mass system: Agilent 6460 QQQ
   b. Ion source: ESI Positive mode
   c. Gas temp (°C): 290
   d. Gas flow (L/min): 12
   e. Nebulizer (psig): 45
   f. Sheath gas temp (°C): 300
   g. Sheath gas flow (L/min): 8
   h. Capillary (V): 3000
   i. Nozzle voltage (V): 2000
   j. MRM transitions:

TABLE 1

| Cpd Name | Prec Ion | Prod Ion | Frag (V) | CE (V) | Cell Acc (V) | Dwell time |
|---|---|---|---|---|---|---|
| PhIP | 225.1 | 210 | 380 | 34 | 5 | 20 msec |
| | | 139.9 | 380 | 45 | 5 | 20 msec |
| PhIP-d3 | 228.1 | 210 | 380 | 34 | 5 | 20 msec |
| | | 140 | 380 | 45 | 5 | 20 msec |
| MelQx | 214.1 | 199.1 | 380 | 30 | 5 | 20 msec |
| | | 130.9 | 380 | 45 | 5 | 20 msec |
| MelQx-d3 | 217.1 | 199.1 | 380 | 34 | 5 | 20 msec |
| | | 131.1 | 380 | 42 | 5 | 20 msec |

### 2. Enzyme Method

To establish the optimal pH condition for hydrolase, 1 ml of 0.2 M sodium acetate buffer (pH 5.0) was added to 1 ml of urine sample. In addition, 4 µl of internal standards (IS, 126.75 µg/L of PhIP-d₃ and 125 µg/L of MeIQx-d₃), and 150 µl (12,750 U) of β-glucuronidase, which is a hydrolase derived from Helix pomatia, were added to the samples, followed by shaking incubation in a water bath at 37° C. for 5 hours.

After the incubation, liquid-liquid extraction, dry, re-dissolving, and filtering was performed in the same manner as in the strong alkali method, thereby was subjected to LC/MS/MS analyses under the same conditions as in the above experiment.

### Strong Acid Method

One hundred µl of 37% HCl (10.15 M) was added to 1 ml of urine sample with 4 µl of the internal standards (IS) and the sample were heated at 70° C. for 3 hours. Then, neutralization was performed with 200 µl of 10N NaOH.

Thereafter, liquid-liquid extraction, dry, re-dissolving, and filtering was performed in the same manner as in the strong alkali method, thereby was subjected to LC/MS/MS analyses under the same conditions as in the above experiment.

### 4. Comparison of LC/MS/MS Results

After urine preparation with the above methods, the analyses with LC/MS/MS were performed to compare quantitative values of PhIP and MeIQx, colorectal cancer-carcinogens in urine.

As a result, the alkali method showed the highest values of PhIP and MeIQx among the methods (TABLE 2).

TABLE 2

| | PhIP | PhIP-d3 | MeIQx | MeIQx-d3 |
|---|---|---|---|---|
| Strong alkali method | 180.45 | 13565.31 | 1251.02 | 3867.24 |
| Enzymatic method | 106.03 | 1842.29 | 381.17 | 1368.04 |
| Strong acid method | 33.55 | 57.17 | 137.61 | 57.17 |

As the strong alkali method showed more obvious performance for the analyses of PhIP and MeIQx than the hydrolysis methods, i.e., enzyme or strong acid methods, the strong alkali hydrolysis and liquid-liquid extraction method may provide a non-invasive and effective diagnoses with a biological sample for early prevention from colorectal cancer.

Although specific parts of the present disclosure have been described in detail above, it is clear for those skilled in the art that these specific descriptions are merely preferred example embodiments, and the scope of the present disclosure is not limited thereto. Accordingly, the substantial scope of the present disclosure is defined by the appended claims and equivalents thereof.

What is claimed is:

1. A method for providing information for internal dose of colorectal cancer-carcinogens, the method comprising:
Collection of a non-invasive biological sample from a subject;
Preparation of the collected sample with strong alkali hydrolysis and liquid-liquid extraction; Analyses of the HCAs in the prepared sample.

2. The method of claim 1, wherein the biological sample is selected from the group consisting of non-invasive biospecimens, such as urine, blood, saliva, and some tissues.

3. The method of claim 1, wherein the collected sample is prepared with the strong alkali in an amount of 5 to 15 parts by weight based on 100 parts by weight of the sample.

4. The method of claim 1, wherein the strong alkali is selected from the group consisting of sodium hydroxide (NaOH) and potassium hydroxide (KOH).

5. The method of claim 1, wherein the performing of the liquid -liquid extraction comprises the use of dichloromethane (CH₂Cl₂) as a specific extraction solvent.

6. The method of claim 1, wherein the HCAs mean one or at least two selected ones from the group consisting of 2-amino-1-methyl-6-phenylimidazo[4-5-b]pyridine (PhIP) and 2-amino-3,8-dimethylimidazo[ 4,5-f]quinoxaline (MeIQx).

7. The method of claim 1, wherein the levels of the HCAs is quantified with liquid chromatography-mass spectrometry (LC/MS/MS).

8. The method of claim 1, wherein a method comprises to serve the information for preemptive diagnoses for exposure to colorectal cancer-carcinogens and to predict that the risk of colorectal cancer is higher in the people with high levels of the HCAs than in the others.
